(19) United States
(12) Patent Application Publication
(10) Pub. No.: US 2014/0142450 A1
(43) Pub. Date: May 22, 2014

(54) SLEEP APNEA DETECTION SYSTEM AND METHOD

(71) Applicant: National Chiao Tung University, Hsinchu (TW)

(72) Inventors: Wai-Chi Fang, Hsinchu (TW); Hsiao-Yu Chen, Hsinchu (TW); Teng-Chieh Huang, Hsinchu (TW)

(73) Assignee: NATIONAL CHIAO TUNG UNIVERSITY, Hsinchu (TW)

(21) Appl. No.: 13/897,867

(22) Filed: May 20, 2013

(30) Foreign Application Priority Data
Nov. 19, 2012 (TW) 101143045

(51) Int. Cl. A61B 5/00; A61B 5/0456

(52) U.S. Cl. CPC A61B 5/4818; A61B 5/0456
USPC 600/521

(57) ABSTRACT

The invention provides a sleep apnea detection system and method. The system includes a detecting module, a processing module, a converting module and a determining module. The detecting module detects a plurality of peak time points of R-waves in an ECG (electrocardiograph) signal. The processing module calculates areas of the R-waves at a predetermined time range based on the peak time points, so as to produce a plurality of first R-wave area signals based on the areas and generate an EDR (ECG Derived Respiration) signal based on the peak time points and the first R-wave area signals. The converting module converts the EDR signal to a frequency signal. The determining module determines whether a maximum peak frequency of the frequency signal is at a first frequency segment or a second frequency segment to determine the frequency signal being an apnea signal or a normal breathing signal.

### BACKGROUND OF THE INVENTION

1. Field of the Invention

This invention relates to a detection of sleep apnea, and more particularly, to a sleep apnea detection system and method.

2. Description of Related Art

Sleep apnea is defined as having cessation in breathing, where each cessation could last from a few seconds to minutes, or shallow breaths during sleep. Sleep apnea can be classified into three types: central sleep apnea (CSA), obstructive picture of the occurrence and elapse time of apnea during sleep apnea (OSA), and mixed apnea.

Central sleep apnea results from the failure of brain to signal breathing command to the muscle, obstructive sleep apnea is caused by the limitation on air flow, which often results in snoring or choking during sleep, and mixed apnea is a combination of central sleep apnea and obstructive sleep apnea. Obstructive sleep apnea is the most common type of sleep apnea.

The current apnea detection systems such as that disclosed in U.S. 2003/0055348A1 is required to simultaneously determine an ECG (electrocardiograph) signal and an EDR (ECG derived respiration) signal, and detecting procedures and algorithms are more complex. For example, calculating PR (P-wave˜R-wave) segment and power density of the ECG signal are required, and therefore the current apnea detection systems are not real-time detecting systems.

Furthermore, in US 2006/0079802A1, it is required to calculate the chest impedance and use devices such as an impedance sensor and a movement sensor.

Therefore, in order to solve the shortcomings of the above conventional technique, a simple detecting procedure, hardware, software, and algorithms for detecting the occurrence of the sleep apnea are provided.

### SUMMARY OF THE INVENTION

The present invention provides a sleep apnea detection system and method. A plurality of peak time points of R-waves in an ECG (electrocardiograph) signal are first detected. Areas of the plurality of R-waves are calculated, and R-wave area signals are produced to generate an EDR signal. The maximum peak and frequency of the frequency signal are simultaneously determined, and then the frequency signal being an apnea signal, a normal breathing signal or a mixed signal is determined. Therefore, the real-time detection of the occurrence of the sleep apnea at a (within 1 minute) is achieved by simple detecting procedure, hardware, software, and algorithms without using additional detection instruments or manual interpretation. Hence, the diagnostic procedures are significantly reduced, and the detecting efficiency is improved by the present invention.

The invention provides a sleep apnea detection system comprising a detecting module, a processing module, a converting module and a determining module. The detecting module detects a plurality of peak time points of R-waves in an ECG (electrocardiograph) signal. The processing module calculates areas of the R-waves at a predetermined time range based on the peak time points, so as to produce a plurality of first R-wave area signals based on the areas and generate an EDR (ECG derived respiration) signal based on the peak time points and the first R-wave area signals. The converting module converts the EDR signal to a frequency signal. The determining module determines whether a maximum peak frequency of the frequency signal is at a first frequency segment or a second frequency segment to determine the frequency signal being an apnea signal or a normal breathing signal.

The invention also provides a method for detecting a sleep apnea, comprising detecting peak time points of a plurality of R-waves of a ECG signal; calculating areas of the plurality of R-waves at a predetermined time range based on the peak time points; producing a plurality of first R-wave area signals based on the areas; generating an EDR (ECG derived respiration) signal based on the peak time points and the plurality of first R-wave area signals; converting the EDR signal to a frequency signal; and determining whether a maximum peak frequency of the frequency signal is at a first frequency segment or a second frequency segment to determine the frequency signal being an apnea signal or a normal breathing signal.

### BRIEF DESCRIPTION OF DRAWINGS

The invention can be more fully understood by reading the following detailed description of the preferred embodiments, with reference made to the accompanying drawings, wherein

FIG. 1 is a schematic block diagram of a sleep apnea detection system according to the present invention;

FIG. 2 is a waveform diagram showing ECG signal according to the present invention;

FIG. 3 is a schematic diagram showing second R-wave area signals produced between first R-wave area signals using a linear interpolation according to the present invention;

FIG. 4A is a waveform diagram of generating EDR signal based on the first R-wave area signals of first embodiment according to the present invention;

FIG. 4B is a waveform diagram of the EDR signal of FIG. 4A generating consecutive signal according to the present invention;

FIG. 4C is a waveform diagram showing the consecutive signal of FIG. 4B converted to frequency signal and determined as an apnea signal according to the present invention;

FIG. 5A is a waveform diagram showing that the EDR signal is generated based on the first R-wave area signals according to the second embodiment of the present invention;

FIG. 5B is a waveform diagram of the EDR signal of FIG. 5A generating consecutive signal according to the present invention;

FIG. 5C is a waveform diagram showing the consecutive signal of FIG. 5B converted to frequency signal and determined as a normal breathing signal according to the present invention;

FIG. 6A is a waveform diagram of the ECG signal having noise according to the third embodiment of the present invention;

FIG. 6B is a waveform diagram showing that the EDR signal is generated based on the first R-wave area signals produced by the ECG signal of FIG. 6A according to the present invention;

FIG. 6C is a waveform diagram of the EDR signal of FIG. 6B generating consecutive signal according to the present invention;

FIG. 6D is a waveform diagram showing the consecutive signal of FIG. 6C converted to frequency signal and determined as a mixed signal according to the present invention;

FIG. 7 is a schematic diagram of accuracy in relation to the normal breathing signal and the apnea signal at different time length according to the present invention; and

FIG. 8 illustrates a schematic flow chart of a sleep apnea detecting method according to the present invention.

### DETAILED DESCRIPTION OF THE INVENTION

The following illustrative embodiments are provided to illustrate the disclosure of the present invention, these and other advantages and effects can be apparently understood by those in the art after reading the disclosure of this specification. The present invention can also be performed or applied by other different embodiments. The details of the specification may be on the basis of different points and applications, and numerous modifications and variations can be devised without departing from the spirit of the present invention.

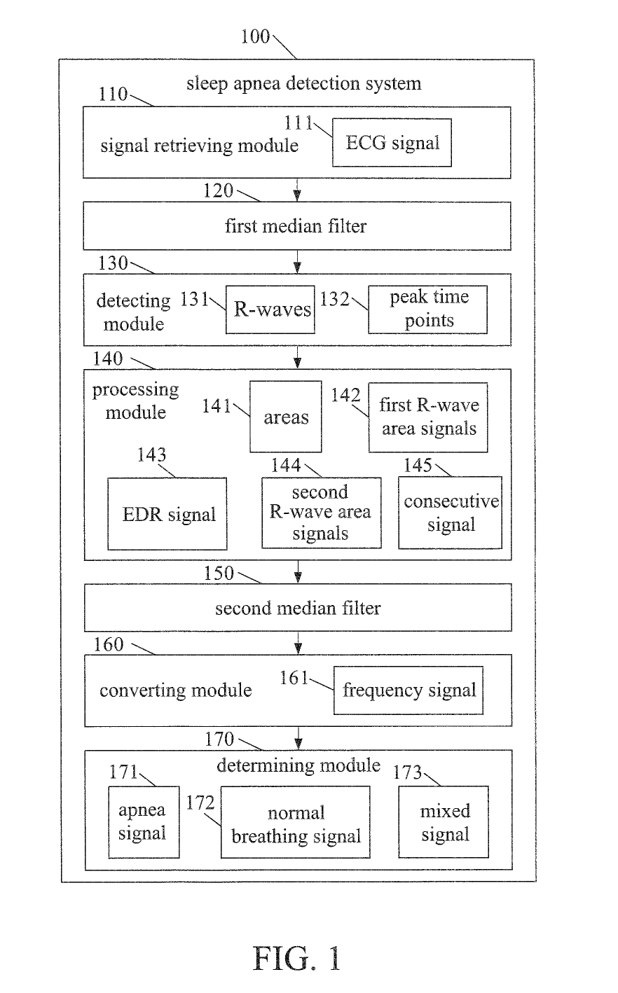

FIG. 1 illustrates a schematic block diagram of a sleep apnea detection system according to the present invention. As shown in FIG. 1, the sleep apnea detection system 100 uses the impact of the expansion or reduction of the chest caused by breathing on the ECG signal 111, calculates areas 141 of the R-waves 131 and generates the EDR signal 143. Then, the judgment is performed in response to the modulation of the EDR signal 143 according to the more intense chest movement resulting from airway obstruction caused by sleep apnea.

Since the chest movement caused by the airway obstruction is relatively longer in cycle and larger in amplitude than the general normal breathing, it can be determined by determining the frequency signal 161 of the EDR signal 143 whether the sleep apnea occurs.

The sleep apnea detection system 100 may detect the OSA, the CSA, or the mixed type of apnea, and may include a signal retrieving module 110, a first median filter 120, a detecting module 130, a processing module 140, a second median filter 150, a converting module 160 and a determining module 170.

The signal retrieving module 110 is used for retrieving the ECG signal 111 of the predetermined time frame at each time interval, for example, retrieving the ECG signal 111 of 1 minute at every 15 seconds. The signal retrieving module 110 may be a signal retrieving program, a signal retrieving software or a signal receiving module.

The first median filter 120 is used for filtering drifting baseline or drifting baseline or negative singularity of the ECG signal 111.

The detecting module 130 is used for detecting a plurality of peak time points 132 of the R-waves 131 in the ECG signal 111 based on a wavelet transform detecting method. The wavelet transform detecting method uses the quadratic spline for a mother wavelet function. The detecting module 130 may be a detector, a detecting program or a detecting software.

The processing module 140 is used for calculating areas 141 of the plurality of R-waves 131 at a predetermined time range based on the peak time points 132 to produce a plurality of first R-wave area signals 142 based on the areas 141 and generate the EDR signal 143 based on the peak time points 132 and the first plurality of R-wave area signals 142. The processing module 140 may be a processor, a processing program or a processing software.

The processing module 140 may also adjust extrema of the plurality of first R-wave area signals 142 and produce a plurality of second R-wave area signals 144 between the plurality of first R-wave area signals 142 based on a linear interpolation, such that the EDR signal 143 generates a consecutive signal 145.

The second median filter 150 is used for filtering the drifting baseline of the EDR signal 143 to strengthen the consecutive signal 145.

The converting module 160 is used for converting the EDR signal 143 to a frequency signal 161 based on a fast Fourier transform (FFT) method. The converting module 160 may be a converting program, a converting software, a converter or a processor.

The determining module 170 is used for determining whether a maximum peak frequency of the frequency signal 161 is at a first frequency segment or a second frequency segment so as to determine the frequency signal 161 being an apnea signal 171 or a normal breathing signal 172

When the maximum peak frequency is located at the first frequency segment, the determining module 170 further compares the maximum peak with a predetermined threshold. When the maximum peak is greater than the threshold, the determining module 170 determines that the frequency signal 161 is a apnea signal 171, and when the maximum peak is less than the threshold, the determining module 170 determines that the frequency signal 161 is a mixed signal 173, which is the normal breathing signal with noise. When the maximum peak frequency is located in the second frequency segment, the determining module 170 determines that the frequency signal 161 is the normal breathing signal 172. The determining module 170 may be a determining program or a processor.

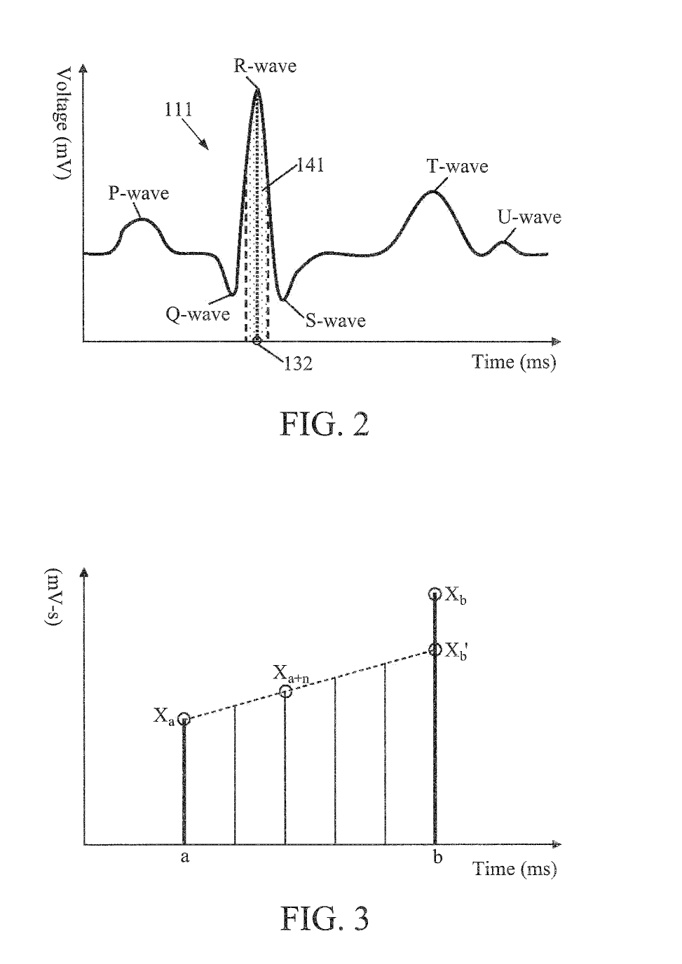

FIG. 2 illustrates a waveform diagram relating to ECG signal according to the present invention. As shown in FIG. 2, the single pulse wave of the ECG signal 111 is divided into P wave, Q wave, R-wave S-wave, T wave and U wave, wherein the R-wave has a maximum peak.

As shown in FIGS. 1 and 2, the detecting module 130 is used for detecting a plurality of peak time points 132 of the R-waves 131 in the ECG signal 111 based on a wavelet transform detecting method. The wavelet transform detecting method uses the quadratic spline for a mother wavelet function.

The processing module 140 is used for calculating areas 141 of the plurality of R-waves 131 at a predetermined time range based on the peak time points 132. For example, an integration is carried out on the R-waves 131 based on the peak time points 132 before or after the time of 50 ms to calculate the areas 141 of the R-waves 131.

FIG. 3 illustrates a schematic diagram of producing second R-wave area signals between first R-wave area signals using a linear interpolation according to the present invention.

As shown in FIG. 3, the extrema of the plurality of first R-wave area signals (such as Xₐ, X_{b}) is adjusted first. For example, the extrema of the first R-wave area signals X_{b} is adjusted as follows:

$$( 1 - α ) {X}_{a} , \\ {X}_{b} ≧ ( 1 + α ) {X}_{a}$$

$${X}_{b}^{′} = {X}_{b} , \\ ( 1 + α ) {X}_{a} > {X}_{b} > ( 1 - α ) {X}_{a} \\ ( 1 + α ) {X}_{a} , {X}_{b} ≦ ( 1 - α ) {X}_{a}$$

wherein Xₐ is the first R-wave area signal, X_{b} is the next first R-wave area signal, X_{b}′ is the adjusted first R-wave area signal. a is the peak time point of the first R-wave area signals Xₐ, and b is the peak time point of the first R-wave area signals X_{b}. α is an adjustment value, such as 0.05 or other value.

For example, when α is 0.05, the above formula means that when the first R-wave area signal Xb is greater than or equal to 1.05 times of the first R-wave area signal Xₐ, the first R-wave area signal X_{b}′ is equal to 0.95 times of the first R-wave area signals Xₐ, for lowering the extrema of the first R-wave area signal X_{b}.

Conversely, when the first R-wave area signals X_{b} is less than or equal to 0.95 times of the first R-wave area signals Xₐ, the first R-wave area signals X_{b}′ is equal to 1.05 times of the first R-wave area signals Xₐ, for raising the extrema of the first R-wave areas signal X_{b}.

Furthermore, when the first R-wave area signals X_{b} is ranged from 0.95 to 1.05 times of the first R-wave area signals Xₐ, the first R-wave area signals X_{b}′ is equal to the first R-wave area signal X_{b}, for maintaining the extrema of the first R-wave area signals X_{b} unchanged.

Therefore, the present invention avoids drastic change of the first R-wave area signals (such as Xₐ, X_{b}) interfering the detect results, and then focuses on the trend of the EDR signal.

After the extrema of the first R-wave area signals X_{b} is completely adjusted, the plurality of second R-wave area signals Xₐ₊ₙ are produced between the first R-wave area signals Xₐ and X_{b} based on the linear interpolation, and then the EDR signal generates consecutive signal. The formula of linear interpolation is as follows:

Xₐ₊ₙ=Xₐ+(X_{b}′−Xₐ)*n/(b−a), n=1,2, . . . ,b−a

wherein Xₐ₊ₙ is the nth second R-wave area signal counted from the first R-wave area signals Xₐ, Xₐ is the first R-wave area signal, X_{b}′ is the adjusted first R-wave area signal, a is the peak time points of the first R-wave area signal Xₐ, b is the peak time points of the first R-wave area signal X_{b}, and n is a positive integer.

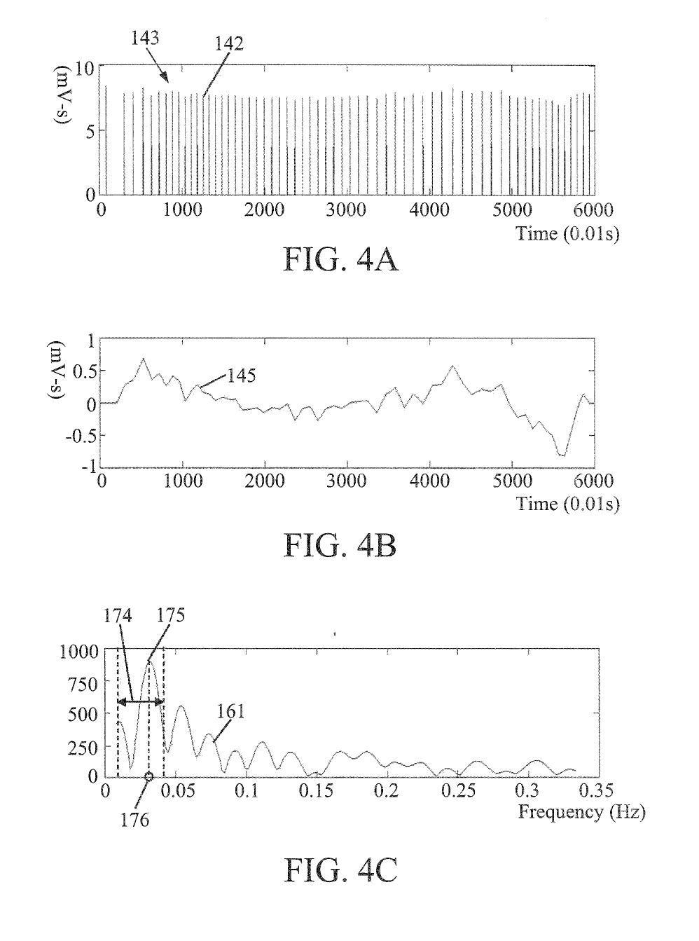

FIG. 4A illustrates a waveform diagram of generating EDR signal based on the first R-wave area signals according to the first embodiment of the present invention. FIG. 4B illustrates a waveform diagram of the EDR signal of FIG. 4A generating consecutive signal according to the present invention. FIG. 4C illustrates a waveform diagram showing the consecutive signal of FIG. 4B converted to frequency signal and determined being an apnea signal according to the present invention.

As shown in FIG. 4A, as FIG. 2, the areas 141 of the plurality of R-waves of the ECG signal 111 are calculated, a plurality of first R-wave area signals 142 are produced based on the areas 141, and the EDR signal 143 is generated based on the wave peak time points 132 and the first R-wave area signals 142 of the R-waves.

As shown in FIG. 4B, the extrema of the plurality first R-wave area signals 142 in FIG. 4A is adjusted, a plurality of second R-wave area signals (not shown) between the first R-wave area signals 142 are produced based on the linear interpolation, and the EDR signal 143 generates the consecutive signal 145.

As shown in FIG. 4C, the consecutive signal 145 is converted to a frequency signal 161, and it is determined whether the frequency 176 of the maximum peak 175 of the frequency signal 161 is in the first frequency segment 174 or the second frequency segment (not shown) in order to determine the frequency signal 161 being the apnea signal or the normal breathing signal.

In the first embodiment, the frequency 176 of the maximum peak 175 of the frequency signal 161 is approximately 0.03 Hz, and is located in the first frequency segment 174, i.e. frequency of 0.01 to 0.04 Hz; and the maximum peak 175 is approximately 900, and more than the predetermined threshold (such as 400). It is thus determined that the frequency signal 161 is the apnea signal.

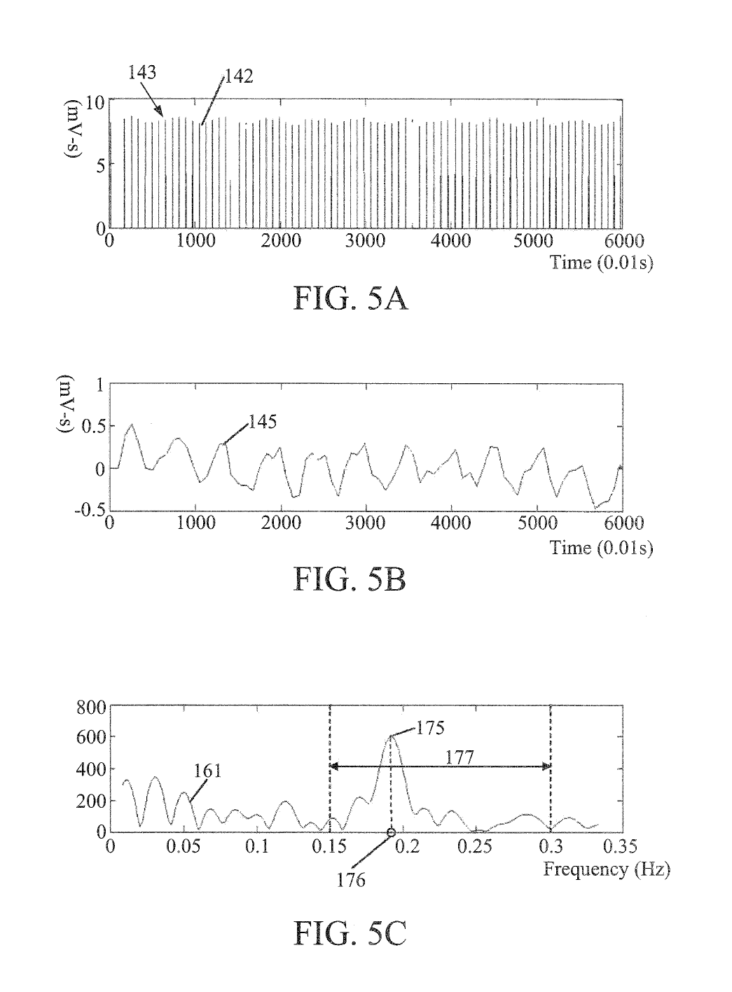

FIG. 5A illustrates a waveform diagram of generating EDR signal based on the first R-wave area signals according to the second embodiment of the present invention. FIG. 5B illustrates a waveform diagram of the EDR signal of FIG. 5A generating consecutive signal according to the present invention. FIG. 5C illustrates a waveform diagram showing the consecutive signal of FIG. 5B converted to the frequency signal and determined being a normal breathing signal according to the present invention.

The generation of the EDR signal, the consecutive signal and the frequency signal in the second embodiment is the same as that in the first embodiment of FIGS. 4A to 4C, and is thus not repeatedly described. The differences of the second embodiment from the first embodiment are illustrated as follows.

In FIG. 5C, the frequency 176 of the maximum peak 175 of the frequency signal 161 is approximately 0.19 Hz, and is located in the second frequency segment 177, i.e. frequency of 0.15 to 0.3 Hz. It is determined that the frequency signal 161 is the normal breathing signal.

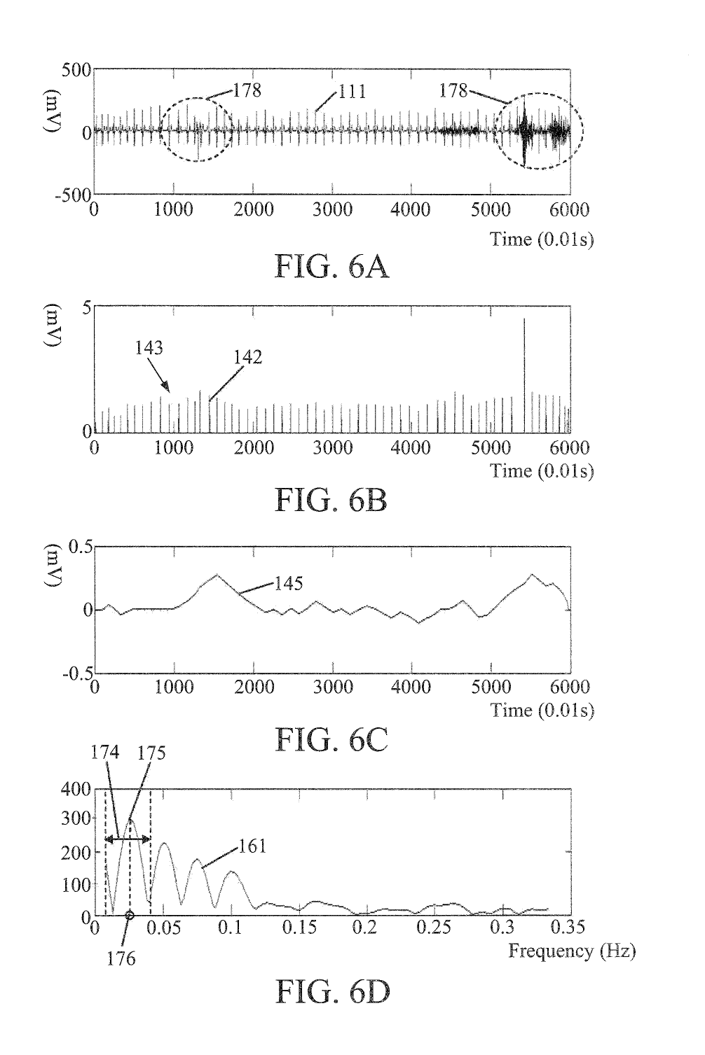

FIG. 6A illustrates a waveform diagram of the ECG signal having noise according to the third embodiment of the present invention. FIG. 6B illustrates a waveform diagram of generating the EDR signal based on the first R-wave area signals produced by the ECG signal of FIG. 6A according to the present invention. FIG. 6C illustrates a waveform diagram of the EDR signal of FIG. 6B generating consecutive signal according to the present invention. FIG. 6D illustrates a waveform diagram showing the consecutive signal of FIG. 6C converted to frequency signal and determined being a mixed signal according to the present invention.

The generation of the EDR signal, the consecutive signal and the frequency signal in the third embodiment is the same as that in the first embodiment of FIGS. 4A to 4C. and it is thus not repeatedly described. The differences of the third embodiment from the second embodiment are illustrated as follows.

In FIG. 6A, the ECG signal 111 has a noise 178.

In FIG. 6D, the frequency 176 of the maximum peak 175 of the frequency signal 161 is approximately 0.03 Hz, and is located in the first frequency segment 174, i.e. frequency of 0.01 to 0.04 Hz. It is determined that the frequency signal 161 may be the apnea signal.

However, due to the ECG signal 111 has the noise 178, when the frequency signal 161 is determined as the apnea signal or the normal breathing signal, it needs to determine whether the maximum peak 175 is greater than a predetermined threshold (eg, 400). If so, the frequency signal 161 is determined as the apnea signal. If not, the maximum peak 175 is less than the threshold, and it is determined that the frequency signal 161 is a mixed signal, which is the normal breathing signal with the noise.

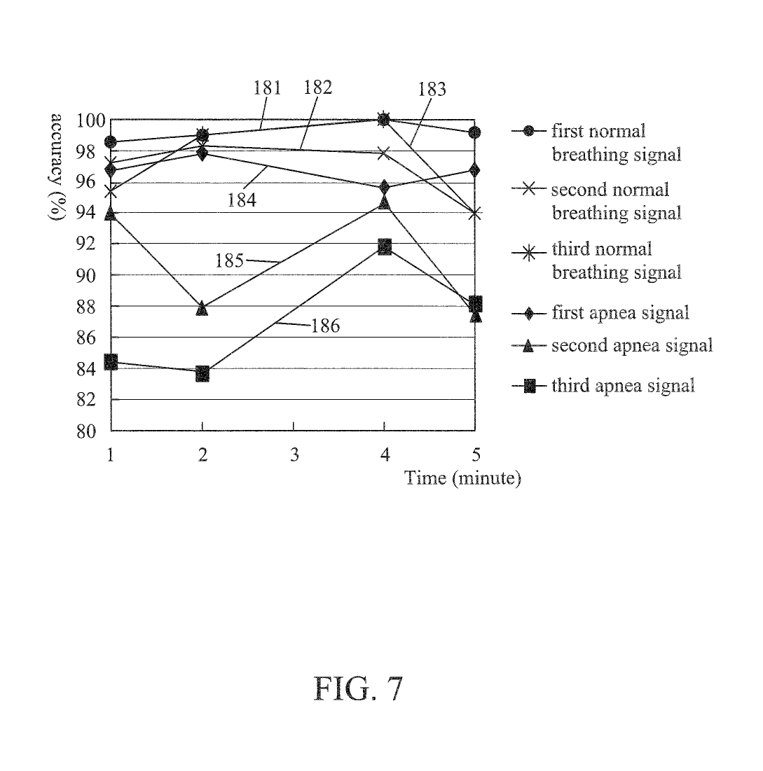

FIG. 7 illustrates a schematic diagram of accuracy in relation to the normal breathing signal and the apnea signal at different time length according to the present invention.

As shown, accuracy 181 of the first Normal breathing signal, accuracy 182 of the second normal breathing signal, accuracy 183 of the third normal breathing signal, accuracy 184 of first apnea signal, accuracy 185 of second apnea signal and accuracy 186 of third apnea signal are respectively detected at different time length. No matter the time length is 1 minute, 2 minutes, 4 minutes, or 5 minutes, accuracy of the detection result is more than 80%. The accuracy of the detection result is not affected by the length of time.

In other words, the present invention can be used as the real-time detection of sleep apnea, wherein the ECG signal is detected for 1 minute every 15 seconds, so as to real-time determine whether the patient has the sleep apnea.

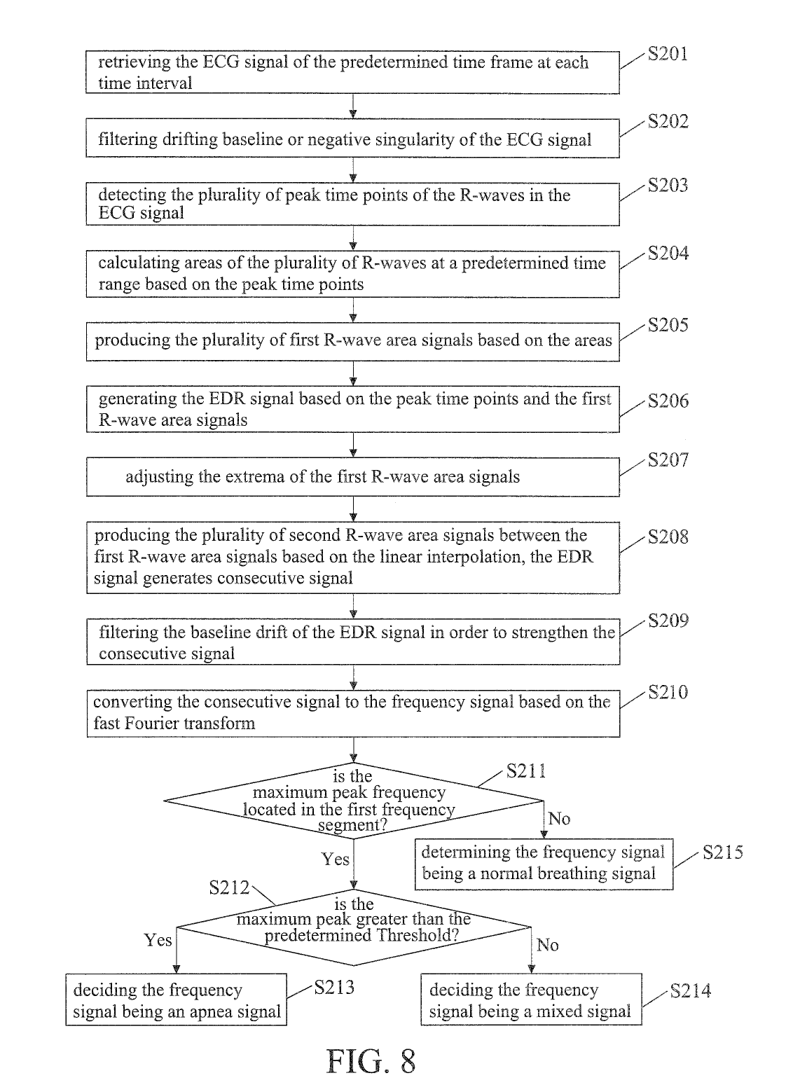

FIG. 8 illustrates a schematic flow chart of a sleep apnea detecting method according to the present invention. As shown, the sleep apnea detecting method may include the following steps:

In step S201, the signal retrieving module retrieves the ECG signal of the predetermined time frame at each time interval. For example, the signal retrieving module retrieves the ECG signal for 1 minute at every 15 seconds. The procedure then goes to step S202.

In step S202, the first median filter filters drifting baseline or drifting baseline or negative singularity of the ECG signal. The procedure then goes to step S203.

In step S203, the detecting module detects the plurality of peak time points of the R-waves in the ECG signal. The procedure then goes to step S204.

In step S204, the processing module calculates areas of the plurality of R-waves at a predetermined time range. The procedure then goes to step S205.

In step S205, the processing module produces the plurality of first R-wave area signals based on the areas. The procedure then goes to step S206.

In step S206, the processing module generates the EDR signal based on the peak time points and the first R-wave area signals. The procedure then goes to step S207.

In step S207, the processing module adjusts the extrema of the first R-wave area signals. The procedure then goes to step S208.

In step S208, the processing module produces the plurality of second R-wave area signals between the first R-wave area signals based on the linear interpolation, and the EDR signal generates a consecutive signal. The procedure then goes to step S209.

In step S209, the second median filters the baseline drift of the EDR signal in order to strengthen the consecutive signal. The procedure then goes to step S210.

In step S210, the converting module converts the consecutive signal to the frequency signal based on the fast Fourier transform. The procedure then goes to step S211.

In step S211, the determining module determines whether the maximum peak frequency of the frequency signal is located in the first frequency segment. If so, the procedure then goes to step S212. If not, the maximum peak frequency is located in the second frequency segment, and the first frequency segment is less than the second frequency segment. The procedure then goes to step S215.

In step S212, the determining module determines whether the maximum peak is greater than the predetermined threshold. If so, the procedure then goes to step S213.

If not, the procedure then goes to step S214.

In step S213, the determining module determines the frequency signal being an apnea signal.

In step S214, the determining module determines the frequency signal being a mixed signal, namely the normal breathing signal with noise.

In step S215, the determining module determines the frequency signal being a normal breathing signal.

The foregoing descriptions of the detailed embodiments are only illustrated to disclose the features and functions of the present invention and not restrictive of the scope of the present invention. It should be understood to those in the art that all modifications and variations according to the spirit and principle in the disclosure of the present invention should fall within the scope of the appended claims.

What is claimed is:

1. A sleep apnea detection system, comprising:
a detecting module for detecting a plurality of peak time points of R-waves in an ECG (electrocardiograph) signal;
a processing module for calculating areas of the plurality of R-waves at a predetermined time range based on the peak time points to produce a plurality of first R-wave area signals based on the areas and generate an EDR (ECG derived respiration) signal based on the peak time points and the first plurality of R-wave area signals;
a converting module for converting the EDR signal to a frequency signal; and
a determining module for determining whether a maximum peak frequency of the frequency signal is at a first frequency segment or a second frequency segment to determine the frequency signal being an apnea signal or a normal breathing signal.

2. The sleep apnea detection system of claim 1, further comprising a signal retrieving module for retrieving the ECG signal of the predetermined time frame at each time interval.

3. The sleep apnea detection system of claim 1, further comprising a first median filter for filtering drifting baseline or negative singularity of the ECG signal.

4. The sleep apnea detection system of claim 1, wherein the processing module further adjusts extrema of the plurality of first R-wave area signals and produces a plurality of second R-wave area signals between the plurality of first R-wave area signals based on a linear interpolation, such that the EDR signal generates a consecutive signal.

5. The sleep apnea detection system of claim 4, further comprising a second median filter for filtering the drifting baseline of the EDR signal to strengthen the consecutive signal, wherein the converting module converts the consecutive signal to the frequency signal based on a fast Fourier transform method.

6. The sleep apnea detection system of claim 1, when the maximum peak frequency is located at the first frequency segment, the determining module further compares the maximum peak with a predetermined threshold, when the maximum peak is greater than the threshold, the determining module determines that the frequency signal is a apnea signal, and when the maximum peak is less than the threshold, the determining module determines that the frequency signal is a mixed signal.

7. The sleep apnea detection system of claim 1, wherein the first frequency segment is less than the second frequency segment, when the maximum peak frequency is located at the second frequency segment, the determining module determines that the frequency signal is the normal breathing signal.

8. A method for detecting a sleep apnea, comprising:
(1) detecting peak time points of a plurality of R-waves of an ECG signal;
(2) calculating areas of the plurality of R-waves at a predetermined time range based on the peak time points;
(3) producing a plurality of first R-wave area signals based on the areas;
(4) generating an EDR (ECG derived respiration) signal based on the peak time points and the plurality of first R-wave area signals;
(5) converting the EDR signal to a frequency signal; and
(6) determining whether a maximum peak frequency of the frequency signal is at a first frequency segment or a second frequency segment to determine the frequency signal being an apnea signal or a normal breathing signal.

9. The method of claim 8, prior to step (1) further comprising:
(0-1) retrieving the ECG signal of a predetermined time frame at each time interval.

10. The method of claim 9, after step (0-1) further comprising: (0-2) filtering drifting baseline or negative singularity of the ECG signal.

11. The method of claim 8, wherein step (5) further comprises:
(5-1) adjusting extrema of the plurality of first R-wave area signals; and
(5-2) producing a plurality of second R-wave area signals between the plurality of first R-wave area signals based on a linear interpolation, such that the EDR signal generates a consecutive signal.

12. The method of claim 11, wherein after step (5-2) further comprises:
(5-3) filtering the drifting baseline of the EDR signal to strengthen the consecutive signal; and
(5-4) converting the consecutive signal to the frequency signal based on a fast Fourier transform method.

13. The method of claim 8, when it is determined in step (6) that the maximum peak frequency is located at the first frequency segment, further comprising:
(6-1) comparing the maximum peak with a predetermined threshold; and
(6-2) determining the frequency signal being an apnea signal when the maximum peak is greater than the threshold, and determining the frequency signal being a mixed signal when the maximum peak is less than the threshold.

14. The method of claim 8, wherein when it is determined in step (6) that the maximum peak frequency is located at the second frequency segment, the frequency signal is determined as the normal breathing signal.
